(19) United States
(12) Patent Application Publication
(10) Pub. No.: US 2007/0249037 A1
(43) Pub. Date: Oct. 25, 2007

(54) Monitoring of Vitamin K Nutritional Status

(76) Inventor: Taili Tee Thula, Gainesville, FL (US)

Correspondence Address: Beusse Wolter Sanks Mora & Maire, 390 N. ORANGE AVENUE, SUITE 2500, ORLANDO, FL, 32801, US

(21) Appl. No.: 10/591,364

(22) PCT Filed: Mar. 16, 2005
(86) PCT No.: PCT/US05/08896

§ 371 Date: Mar. 22, 2007

Related U.S. Application Data
(60) Provisional application No. 60/553,690, filed on Mar. 16, 2004.

(51) Int. Cl. C12M 1/34; G01N 33/86

(52) U.S. Cl. 435/287.1; 435/288.7; 436/69

(57) ABSTRACT

Disclosed herein is a diagnostic apparatus for determining the level of certain nutrients in a body fluid sample, which are used by clinicians to assess proper dosages of anticoagulants, such as coumarin. Specifically exemplified is a diagnostic apparatus that determines a patient's vitamin K nutritional status by determining the level of vitamin K in a blood or serum sample based on a direct correlation with a concentration of carboxylated osteocalcin in the sample.

### BACKGROUND OF THE INVENTION

The quantification of chemical and biochemical components in colored aqueous fluids, in particular colored biological fluids such as whole blood and urine and biological fluid derivatives such as serum and plasma, is of ever-increasing importance. Important applications exist in medical diagnosis and treatment and in the quantification of exposure to therapeutic drugs, intoxicants, hazardous chemicals and the like. In some instances, the amounts of materials being determined are either so miniscule—in the range of a microgram or less per deciliter—or so difficult to precisely determine that the apparatus employed is complicated and useful only to skilled laboratory personnel. In this case the results are generally not available for some hours or days after sampling. In other instances, there is often an emphasis on the ability of lay operators to perform the test routinely, quickly and reproducibly outside a laboratory setting with rapid or immediate information display.

One common medical test is the measurement of blood glucose levels by diabetics. Current teaching counsels diabetic patients to measure their blood glucose level from two to seven times a day depending on the nature and severity of their individual cases. Based on the observed pattern in the measured glucose levels the patient and physician together make adjustments in diet, exercise and insulin intake to better manage the disease. Clearly, this information should be available to the patient immediately.

Devices for enabling patients to test their own blood are well known in the art. One such device is shown in U.S. Pat. No. 4,552,458, to Lowne, which deals with a compact reflectometer to enable the exposure of a reagent to different light beams, one red and one green. The light beams are folded by a reflecting surface, which redirects the beams through a transparent glass plate onto a reagent strip. Light is reflected back from the strip along a similar folded path onto a detector located in the same plane as the light sources.

Most self-monitoring devices relate to monitoring one's blood glucose concentration levels. Apart from glucose, there are only a few other types of analytes, that are currently self-monitored.

Normal hemostasis is the result of a delicate balance between the processes of clot formation (blood coagulation) and clot dissolution (fibrinolysis). The complex interactions between blood cells, specific plasma proteins and the vascular surface, maintain the fluidity of blood unless injury occurs. Damage to the endothelial barrier lining the vascular wall exposes underlying tissue to these blood components. This in turn triggers a series of biochemical reactions altering the hemostatic balance in favor of blood coagulation which can either result in the desired formation of a hemostatic plug stemming the loss of blood or the undesirable formation of an occlusive intravascular thrombus resulting in reduced or complete lack of blood flow to the affected organ.

Substances which interfere in the process of blood coagulation (anticoagulants) have been demonstrated to be important therapeutic agents in the treatment and prevention of thrombotic disorders (Kessler, C. M. (1991) Chest 99: 97S-112S and Cairns, J. A., Hirsh, J., Lewis, H. D., Resnekov, L., and Theroux, P. (1992) Chest 102: 456S-481S). Blood coagulation problems, if not closely monitored, can be a serious medical problem. If coagulation problems are not properly treated and controlled, thrombi can form which may damage vital organ systems, including the brain and kidneys. Conversely, if too much anticoagulant is prescribed, small internal injuries can, in some cases, result in fatal hemorrhaging. It can be very difficult for the clinician to assess the proper dosage of anticoagulants, due, in part, to the fact that their efficacy is directly related to various nutrients in the blood. The convention protocol for determining the proper dosage of anticoagulants is essentially a trial and error process where a dosage is prescribed, coagulation is later tested, and dosage is altered to try to optimize a desired coagulation. This protocol, and the overall determination of proper dosage does not account for the impact of vitamin K on the efficacy of anticoagulants. Thus, a patient may show a high coagulation count which would indicate the need for a higher dosage. However, because of a transient diet change, the patient's vitamin K levels may be higher than usual at that point in time. This can result in over-prescribing the anticoagulant which is not discovered until the next patient visit, weeks or even months later. Indeed, based on the change in vitamin K nutritional status, it can take several months to assess the proper anticoagulant dosages for a patient.

There is a need in the art for a simple and quick method to assess certain nutritional components of a patient's blood so that dosages of anticoagulants can be determined in an expedient manner.

### DESCRIPTION OF PREFERRED EMBODIMENTS

Accordingly, one object of the present invention is to provide a diagnostic apparatus for determining the levels of certain nutrients in a body fluid sample, which are used by clinicians to assess proper dosages of anticoagulants, such as coumarin. In preferred embodiments, the diagnostic apparatus determines a patient's vitamin K nutritional status by determining the level of vitamin K in a blood or serum sample based on a direct correlation with the concentration of carboxylated osteocalcin in the sample. Those skilled in the art will appreciate that other suitable markers may be used in accord with the teachings of the subject invention.

In a specific aspect, carboxylated osteocalcin is determined through ELISA principles by using an antibody specific for carboxylated osteocalcin. Osteocalcin is a vitamin K-dependent bone calcium binding protein also called bone gla protein (BGP). Particularly, human osteocalcin is a relatively small protein composed of 49 amino acids and having a molecular weight of 5800. This protein is produced from osteoblast, and occupies about 20% of the constituent components of non-collagen protein of the bones. This protein contains gamma-carboxyglutamic acid residues and has a strong affinity for hydroxyapatite, and it is therefore presumed to have an important role in the formation of the bone matrices. Antibodies specific for carboxylated osteocalcin are commercially available, see for example Zymed, Gla-type Osteocalcin EIA Kit (cat No. 99-0054) and Koyama, N. et al. (1991) J. Immunol. Meth. 139, 17. It has been demonstrated that carboxylated osteocalcin is a viable marker to determine nutritional levels of vitamin K. L J Sokoll, S L Booth, M E O'Brien, K W Davidson, K I Tsaioun, and J A Sadowski; “Changes in serum osteocalcin, plasma phylloquinone, and urinary gamma-carboxyglutamic acid in response to altered intakes of dietary phylloquinone in human subjects”; Am J Clin Nutr 65: 779-784; and L J Sokoll, and J A Sadowski; “Comparison of biochemical indexes for assessing vitamin K nutritional status in a healthy adult population”; Am J Clin Nutr 63: 566-573.

In alternative embodiments, Vitamin K nutritional status can be determined by measuring other Vitamin K markers such as, but not limited to, vitamin K dependent or interactive proteins such as prothrombin, protein C, S, and Z, coagulation factors VII, IX, and X.

According to another aspect, the subject invention pertains to a method of determining the proper dosage for an anticoagulant comprising the steps of measuring the vitamin K nutritional status of a patient; and modulating the dosage of an anticoagulant based on the patients Vitamin K nutritional status. Modulation of anticoagulation dosages may comprise increasing dosages, decreasing dosages or maintaining dosages based on the expected effect of certain nutritional values for vitamin K in the patient, as well as a patient's diet patterns. One problem physicians face in determining proper dosage of anticoagulants are aberrant coagulation readings caused by a transient change in a patient's diet. For example, if a patient has consumed vitamin K containing foods proximate to the coagulation reading, clinicians will be compelled to erroneously prescribe a higher dosage of anticoagulant, as the patient's blood will show a high propensity for coagulation. However, when the patient ceases the temporary intake of the Vitamin K, the dosage will ultimately be too high. For this reason, finding the proper dosage of anticoagulants can be challenging and take several months to accomplish. The subject invention will alleviate the unnecessary adjustments to anticoagulant dosages as the clinician will be able to immediately determine the effect of nutritional status on coagulation. This is similar to the adjustment of insulin levels based on a diabetic's diet.

In a specific embodiment, the method comprises measuring the vitamin K nutritional status of a patient by determining a first concentration of carboxylated osteocalcin in the patient's blood at a first point in time; determining a second concentration of carboxylated osteocalcin in the patient's blood at a second point in time; obtaining a vitamin K nutritional value based on said first and second carboxylated osteocalcin concentration; assessing the coagulation of the patient's blood; correlating the coagulation of a patients blood with said vitamin K nutritional value; and determining an optimal dosage of anticoagulant based on said vitamin K nutritional value.

In yet another embodiment, the subject invention relates to an apparatus that is able to quickly assess (preferably less than 5 hours, and more preferably, less than 1 hour, most preferably less than 0.5 hours) a patient's vitamin K nutritional status by producing a signal (e.g. calorimetric, electrical, etc) corresponding to the level of a vitamin K marker in a patient's body fluid sample. Preferably, the apparatus comprises a processor and a monitor so that the signal is processed to create a vitamin K value which is then displayed on the monitor. This aspect can be adapted to be included in currently used self-monitoring devices for assessing glucose levels in the blood.

Furthermore, used in conjunction with the diagnostic apparatus, one aspect of the subject invention relates to a disposable diagnostic reagent unit which engages onto or into the diagnostic apparatus. The disposable diagnostic reagent unit carries reagent material for sensing components of a body fluid sample for qualities such as vitamin K levels. In a preferred aspect, the disposable diagnostic reagent unit comprises the appropriate reagent materials to generate a signal corresponding to carboxylated osteocalcin levels in a blood sample. Further, the diagnostic apparatus is configured to removeably engage the disposable diagnostic reagent unit.

The diagnostic apparatus comprises a housing, a microprocessor and related electromechanical and software components to assess the concentration of the signal provided by the disposable diagnostic unit, and a display (e.g. LCD). Preferably, the housing is an easily transportable weight and size, and further comprises a power source such as batteries. However, the apparatus can be of any suitable size depending on the end-use, e.g., doctor's office or patient self-monitoring. Once the reagent material undergoes a calorimetric, potentiometric, or absorption action proportional to the concentration of the vitamin K marker in the sample, the microprocessor circuitry processes the signal and displays the results on an LCD display. In a preferred embodiment, the diagnostic apparatus comprises a light source and a light sensor, and the reagent material in the disposable diagnostic reagent unit produces a calorimetric signal based on the concentration of carboxylated osteocalcin. The reflectance from the reagent chemistry is converted into a electrical signal, processed and displayed as a vitamin K value.

Preferably, the disposable diagnostic reagent unit is able to simultaneously develop two or more microsamples so as to minimize erroneous readings. Preferably still, the disposable diagnostic reagent unit is able to simultaneously develop five microsamples, wherein the lowest and highest reading are ignored, and an average of the middle three readings is produced.

Immunoassay Chip Embodiment

In a specific embodiment, the disposable reagent unit comprises an immunoassay chip. For example, the immunoassay chip as taught in “Integration of Chemical and Biochemical Analysis Systems into a Glass Microchip” Analytical Sciences, January 2003 Vol. 19 (whose teachings are incorporated by reference) is adapted and configured for removeably engaging the diagnostic apparatus. The diagnostic apparatus comprises a light source that is directed to the developed portion of the immunoassay chip to produce a reflectance, or fluorescence, colorimetric or other signal that is detected by a sensor integrated into the diagnostic apparatus and processed to provide a value of the vitamin K marker in the sample.

In another example, the immunoassay chip as taught in “Enzyme linked immunosorbent assay on a microchip with electrochemical detection” The Royal Society of Chemistry, November 2001, 153-157 (incorporated herein by reference) is adapted for use with the diagnostic apparatus, such that it removeably engages to the diagnostic apparatus. In this case, an electrical signal is produced corresponding to the concentration of the vitamin K marker present in the sample. The electrical signal is processed to provide a value of the vitamin K marker in the sample.

With respect to the immunoassay chips discussed above, various vacuum sources can be applied to facilitate the movement of fluid through the system. For example, a small vacuum pump can be integrated into diagnostic apparatus such that it engages to the outlet of the immunoassay chip system. Alternatively, an evacuated tube can be integrated either in the diagnostic apparatus or disposable diagnostic reagent unit. Given the small amounts of fluid utilized in the system, the vacuum can act as both a vacuum and deposit area for the fluids, i.e., body fluid sample, washes, antibody treatments and development steps. Furthermore, a pad, similar absorption source may be utilized which, through basic capillary action pulls fluids through the system. Those skilled in the are will readily appreciate other alternatives for moving fluid through the system, and be able to make substitutions or modifications in this regard in light of the teachings herein.

According to another aspect, the subject invention pertains to an immunoassay chip comprising microchannels having walls where an antibody specific to carboxylated osteocalcin is bound.

Further still, one aspect of the invention pertains to a disposable diagnostic reagent unit that produces a signal corresponding to the concentration of vitamin K in a body fluid sample, without the need to be engaged with another apparatus. This embodiment resembles conventional pregnancy tests devices wherein a body fluid sample such as blood is disposed in the unit and developed to produce a signal. The signal may be calorimetric wherein concentration is determined as a function of where the color reading fits in predetermined color spectrum. The disposable diagnostic reagent unit may itself include simple, inexpensive electromechanical and software elements, display and/or speaker, and small power source such that it provides the desired information regarding the concentration of vitamin K, or other analyte, in the sample. The disposable diagnostic reagent unit is discarded after use.

### Test Strip Embodiment for Disposable Diagnostic Reagent Unit

Those skilled in the art, in view of the teachings herein, will appreciate that the disposable reagent unit can take the form of a simple test strip similar to that used in conventional self-monitoring systems. For example, the test strip device as disclosed in U.S. Pat. No. 6,352,862 is adapted to produce a signal corresponding to presence of the analyte corresponding to vitamin K concentration, which is recited partially herein and incorporated herein in its entirety, to the extent not inconsistent with the teachings herein. Further, the teachings of the references cited in the '862 patent are incorporated by this reference to the extent not inconsistent with the teachings herein.

Accordingly, one version of the test strip embodiment pertains to an analytical test device incorporating a dry porous carrier to which a liquid sample suspected of containing a Vitamin K marker can be applied indirectly, the device also incorporating a labeled specific binding reagent which is freely mobile in the porous carrier when in the moist state, and an unlabeled specific binding reagent which is permanently immobilized in a detection zone on the carrier material, the labeled and unlabeled specific binding reagents being capable of participating in either a sandwich reaction or a competition reaction in the presence of the Vitamin K marker, in which prior to the application to the device of a liquid sample suspected of containing the marker, the labeled specific binding reagent is retained in the dry state in a macroporous body through which the applied liquid sample must pass en route to the porous carrier material, the labeled specific binding reagent being freely soluble or dispersible in any liquid sample which enters the macroporous body.

Preferably, the dry porous carrier material comprises a chromatographic strip, such as a strip of nitrocellulose. If desired, the nitrocellulose can be backed with moisture impermeable material, such as polyester sheet. Using nitrocellulose as the porous carrier material has considerable advantage over more conventional strip materials, such as paper, because nitrocellulose has a natural ability to bind proteins without requiring prior sensitisation. Specific binding reagents, such as immunoglobulins, can be applied directly to nitrocellulose and immobilized thereon. No chemical treatment is required which might interfere with the essential specific binding activity of the reagent. Unused binding sites on the nitrocellulose can thereafter be blocked using simple materials, such as polyvinylalcohol. Moreover, nitrocellulose is readily available in a range of pore sizes and this facilitates the selection of a carrier material to suit particularly requirements such as sample flow rate. Preferably the nitrocellulose has a pore size of at least one micron. Preferably the nitrocellulose has a pore size not greater than about 20 microns.

In a preferred embodiment of the invention, the labeled specific binding reagent comprises a specific binding reagent attached to a particulate label. Such “direct labels”, e.g. colored latex particles, gold sols, non-metallic colloids, and dye sols, are already known per se. They can be used to produce an instant analytical result without the need to add further reagents in order to develop a detectable signal. They are robust and stable and can therefore be used readily in an analytical device which is stored in the dry state. Their release on contact with an aqueous sample can be modulated, for example by the use of soluble glazes. Preferably, the particulate label is a latex particle, such as a colored latex particle which can be readily visible to the eye if it becomes bound in the detection zone. If desired, the assay result can be read instrumentally, eg. by color reflectance. Alternatively, the latex particle can incorporate a fluorescent compound which can respond to applied electromagnetic energy such as ultraviolet light or visible light, to provide an emitted signal that can be measured instrumentally. For example, upon development, the test strip device could be configured such that it can be engaged to the diagnostic apparatus discussed above. The diagnostic apparatus then senses the level of the Vitamin K marker present in the sample, processes the signal and provides a Vitamin K reading. In a particularly preferred embodiment, the direct label is a colored latex particle of spherical or near-spherical shape and having a maximum diameter of not greater than about 0.5 micron. An ideal size range for such particles is from about 0.05 to about 0.5 microns.

The labeled reagent is preferably incorporated in the macroporous material in bulk, eg. large sheet, form before it is subdivided into individual bodies for use in a testing device of the invention.

After a solution containing the labeled reagent has been allowed to saturate the macroporous material, the macroporous material should be dried, eg. by vacuum or air-drying, or preferably by freeze-drying. Optionally, the solution can also contain a surface active agent, such as a detergent, and/or a glazing material, such as a sugar, e.g. sucrose. The presence of the glazing material appears to enhance release of the labeled reagent and promotes stability of delicate specific binding reagents such as antibodies.

By incorporating the labeled reagent in a separate macroporous body, rather than pre-dosed onto the carrier material that also incorporates the detection zone, the following advantages can be obtained:

(i) Enhanced sensitivity of the test, because a substantial quantity of the liquid sample is able to take up the labeled reagent before migrating through the carrier material to the detection zone, enhancing potential reaction time without significantly increasing overall test time. Also, the liquid which permeates the carrier is of a more uniform and consistent composition. Whereas the test devices as described in our earlier patent application GB 2204398A are primarily, although not exclusively, suited to qualitative assays, those of the present invention are especially suitable for quantitative assays as well as for qualitative assays.

(ii) Enhanced perceived performance of the test. For example, when the device incorporates a carrier strip and the detection zone comprises a line of immobilized reagent, and the label is a visible direct label, a positive result shows up more clearly, with much reduced temporary background caused by the visible labeled reagent being progressively conveyed past the detection zone.

(iii) Ease of manufacture, because the incorporation of the labeled reagent in the separate macroporous body avoids the need to apply the labeled reagent in a special zone in the carrier, which may need careful pre-treatment, as described in our GB 2204398A.

If the assay device is intended to identify more than one analyte in a single sample, the macroporous body can incorporate several labeled specific binding reagents each carrying a different label, eg. having different colors or fluorescent properties. This will facilitate the manufacture of a multiple analyte testing device that detects a vitamin K marker in addition to another analyte.

Ideally, the inacroporous body is in direct moisture-conductive contact with the porous material, and the detection zone on the porous carrier material is spaced away from the region of contact between the porous carrier material and the macroporous body. In such an embodiment, the quantity of liquid sample required to saturate the macroporous body is preferably not less than the quantity of liquid sample capable of being absorbed by the mass of porous carrier material linking the macroporous body and the detection zone. In other words, the liquid capacity of the macroporous body is at least equal to the liquid capacity of the working portion of the porous carrier.

The invention also provides an analytical method in which a device as set forth above is contacted with an aqueous liquid sample suspected of containing the analyte, such that the sample permeates by capillary action via the macroporous body through the porous solid carrier into the detection zone and the labeled reagent migrates therewith to the detection zone, the presence of analyte in the sample being determined by observing the extent (if any) to which the labeled reagent becomes bound in the detection zone.

In one embodiment of the invention, the labeled reagent is a specific binding partner for the analyte. The labeled reagent, the analyte (if present) and the immobilized unlabeled specific binding reagent cooperate together in a “sandwich” reaction. This results in the labeled reagent being bound in the detection zone if analyte is present in the sample. The two binding reagents must have specificities for different epitopes on the analyte.

In another embodiment of the invention, the labeled reagent is either the analyte itself which has been conjugated with a label, or is an analyte analogue, ie a chemical entity having the identical specific binding characteristics as the analyte, and which similarly has been conjugated with a label. In the latter case, it is preferable that the properties of the analyte analogue which influence its solubility or dispersibility in an aqueous liquid sample and its ability to migrate through the moist porous solid phase material should be identical to those of the analyte itself, or at least very closely similar. In this second embodiment, the labeled analyte or analyte analogue will migrate through the porous carrier into the detection zone and bind with the immobilized reagent. Any analyte present in the sample will compete with the labeled reagent in this binding reaction. Such competition will result in a reduction in the amount of labeled reagent binding in the detection zone, and a consequent decrease in the intensity of the signal observed in the detection zone in comparison with the signal that is observed in the absence of analyte in the sample.

In a further alternative embodiment, a Vitamin K marker or analogue is immobilized in the detection zone, and the labeled reagent is specific for the analyte. If an analyte-containing sample is applied to the device, competition between the immobilized and free analyte reduced the extent to which the labeled reagent may become bound in the detection zone.

In a further embodiment of the present invention, the porous carrier is linked via the macro-porous body to a porous receiving member to which the liquid sample can be applied and from which the sample can permeate into the porous carrier. Preferably, the porous carrier and the macroporous body are contained within a moisture-imperneable casing or housing and the porous receiving member extends out of the housing and can act as a means for permitting a liquid sample to enter the housing and reach the porous carrier. The housing should be provided with means, e-g. appropriately placed apertures, which enable the detection zone of the porous solid phase carrier material (carrying the immobilized unlabeled specific binding reagent) to be observable from outside the housing so that the result of the assay can be observed. If desired, the housing may also be provided with further means which enable a further zone of the porous solid phase carrier material to be observed from outside the housing and which further zone incorporates one or more control reagents which enable an indication to be given as to whether the assay procedure has been completed. Preferably the housing is provided with a removable cap or shroud which can protect the protruding porous receiving member during storage before use. If desired, the cap or shroud can be replaced over the protruding porous receiving member, after sample application, while the assay procedure is being performed.

According to a preferred embodiment invention the test strip apparatus comprises a hollow elongated casing containing a dry porous nitrocellulose carrier which communicates indirectly with the exterior of the casing via a bibulous blood receiving member which protrudes from the casing, the porous nitrocellulose carrier and the sample receiving member being linked via a macroporous body such that any sample reaching the porous carrier must first pass through the macroporous body, the sample receiving member and the macroporous body together acting as a reservoir from which blood is released into the porous carrier, the macroporous body containing a highly-specific anti-carboxylated osteocalcin antibody bearing a colored “direct” label, the labeled antibody being freely mobile within the macroporous body and the porous carrier when in the moist state, and in a detection zone on the carrier spatially distant from the macroporous body an highly-specific unlabeled anti-carboxylated osteocalcin antibody which is permanently immobilized on the carrier material and is therefore not mobile in the moist state, the labeled and unlabeled antibodies having specificities for different carboxylated osteocalcin epitopes, the casing being constructed of opaque or translucent material incorporating at least one aperture through which the analytical result may be observed, together with a removable and replaceable cover for the protruding bibulous blood receiving member.

Such devices can be provided as kits suitable for home use, comprising a plurality (e.g. two) of devices individually wrapped in moisture impervious wrapping and packaged together with appropriate instructions to the user.

The porous sample receiving member can be made from any bibulous, porous or fibrous material capable of absorbing liquid rapidly. The porosity of the material can be unidirectional (i.e. with pores or fibres running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used. It can be advantageous to pre-treat the member with a surface-active agent during manufacture, as this can reduce any inherent hydrophobicity in the member and therefore enhance its ability to take up and deliver a moist sample rapidly and efficiently. Porous sample receiving members can also be made from paper or other cellulosic materials, such as nitro-cellulose. Materials that are now used in the nibs of so-called fibre tipped pens are particularly suitable and such materials can be shaped or extruded in a variety of lengths and cross-sections appropriate in the context of the invention. Preferably the material comprising the porous receiving member should be chosen such that the porous member can be saturated with aqueous liquid within a matter of seconds. Preferably the material remains robust when moist, and for this reason paper and similar materials are less preferred in any embodiment wherein the porous receiving member protrudes from a housing. The liquid must thereafter permeate freely from the porous sample receiving member into the macroporous body.

If present, the “control” zone can be designed merely to convey an unrelated signal to the user that the device has worked. For example, the control zone can be loaded with an antibody that will bind to the labeled reagent, e.g. an “anti-mouse” antibody if the labeled reagent is an antibody that has been derived using a murine hybridoma, to confirm that the sample has permeated the test strip. Alternatively, the control zone can contain an anhydrous reagent that, when moistened, produces a colour change or colour formation, e.g. anhydrous copper sulphate which will turn blue when moistened by an aqueous sample. As a further alternative, a control zone could contain immobilized analyte which will react with excess labeled reagent from the first zone. As the purpose of the control zone is to indicate to the user that the test has been completed, the control zone should be located downstream from the detection zone in which the desired test result is recorded. A positive control indicator therefore tells the user that the sample has permeated the required distance through the test device.

The label can be any entity the presence of which can be readily detected. Preferably the label is a direct label, ie. an entity which, in its natural state, is readily visible either to the naked eye, or with the aid of an optical filter and/or applied stimulation, e.g. UV light to promote fluorescence. For example, minute colored particles, such as dye sols, metallic sols (e.g. gold), and colored latex particles, are very suitable. Of these options, colored latex particles are most preferred. Concentration of the label into a small zone or volume should give rise to a readily detectable signal, e.g. a strongly-colored area. This can be evaluated by eye, or by instruments if desired.

Indirect labels, such as enzymes, e.g. alkaline phosphatase and horseradish peroxidase, can be used but these usually require the addition of one or more developing reagents such as substrates before a visible signal can be detected. Hence these are less preferred. Such additional reagents can be incorporated in the porous solid phase material or in the macroporous body, or in the sample receiving member if present, such that they dissolve or disperse in the aqueous liquid sample. Alternatively, the developing reagents can be added to the sample before contact with the porous material or the porous material can be exposed to the developing reagents after the binding reaction has taken place.

Coupling of the label to the specific binding reagent can be by covalent bonding, if desired, or by hydrophobic bonding. Such techniques are commonplace in the art, and form no part of the present invention. In the preferred embodiment, where the label is a direct label such as a colored latex particle, hydrophobic bonding is preferred.

According to this embodiment, it is essential that the labeled reagent migrates with the liquid sample as this progresses to the detection zone. Preferably, the flow of sample continues beyond the detection zone and sufficient sample is applied to the porous carrier material in order that this may occur and that any excess labeled reagent which does not participate in any binding reaction in the detection zone is flushed away from the detection zone by this continuing flow. If desired, an absorbant “sink” can be provided at the distal end of the carrier material. The absorberit sink may comprise, for example, Whatman 3MM chromatography paper, and should provide sufficient absorptive capacity to allow any unbound conjugage to wash out of the detection zone. As an alternative to such a sink it can be sufficient to have a length of porous solid phase material which extends beyond the detection zone.

The presence or intensity of the signal from the label which becomes bound in the detection zone can provide a qualitative or quantitative measurement of analyte in the sample. A plurality of detection zones arranged in series on the porous solid phase material, through which the aqueous liquid sample can pass progressively, can also be used to provide a quantitative measurement of the analyte, or can be loaded individually with different specific binding agents to provide a multi-analyte test.

The immobilized reagent in the detection zone is preferably a highly specific antibody, and more preferably a monoclonal antibody. In the embodiment of the invention involving the sandwich reaction, the labeled reagent is also preferably a highly specific antibody, and more preferably a monoclonal antibody.

Preferably the porous carrier material is in the form of a strip or sheet to which during manufacture of the device, one or more reagents can be applied in spacially distinct zones. During use, the liquid sample is allowed to permeate through the sheet or strip from one side or end to another.

If desired, a device according to the invention can incorporate two or more discrete bodies of porous solid phase carrier material, e.g. separate strips or sheets, each carrying immobilized reagents. These discrete bodies can be arranged in parallel, for example, such that a single application of liquid sample to the device initiates sample flow in the discrete bodies simultaneously. The separate analytical results that can be determined in this way can be used as control results, or if different reagents are used on the different carriers, the simultaneous determination of a plurality of analytes in a single sample can be made. Alternatively, multiple samples can be applied individually to an array of carriers and analysed simultaneously.

The material comprising the porous solid phase is preferably nitrocellulose. This has the advantage that proteinaceous reagents, such as an antibody, in the detection zone can be immobilized firmly without prior chemical treatment. If the porous solid phase material comprises paper, for example, the immobilization of an antibody in the second zone needs to be performed by chemical coupling using, for example, CNBr, carbonyldiimidazole, or tresyl chloride.

Following the application of the specific biding reagent to the detection zone, the remainder of the porous solid phase material should be treated to block any remaining binding sites elsewhere. Blocking can be achieved by treatment with protein (e.g. bovine serum albumin or milk protein), or with polyvinylalcohol or ethanolamine, or any combination of these agents, for example. Between these process steps the porous solid phase carrier material should be dried.

Preferably the porous solid phase material is nitrocellulose sheet having a pore size of at least about 1 micron, even more preferably of greater than about 5 microns, and yet more preferably about 8-12 microns. Very suitable nitrocellulose sheet having a nominal pore size of up to approximately 12 microns, is available commercially from Schleicher and Schuell GmbH.

Preferably, the nitrocellulose sheet is “backed”, e.g. with plastics sheet, to increase its handling strength. This can be manufactured easily by forming a thin layer of nitrocellulose on a sheet of backing material. The actual pore size of the nitrocellulose when backed in this manner will tend to be, lower than that of the corresponding unbacked material.

Alternatively, a pre-formed sheet of nitrocellulose can be tightly sandwiched between two supporting sheets of solid material, e.g. plastics sheets.

It is preferable that the flow rate of an aqueous sample through the porous solid phase material should be such that in the untreated material, aqueous liquid migrates at a rate of 1 cm in not more than 2 minutes, but slower flow rates can be used if desired.

The spatial separation between the macroporous body and the detection zone, and the flow rate characteristics of the porous carrier material, can be selected to allow adequate reaction times during which the necessary specific binding can occur. Further control over these parameters can be achieved by the incorporation of viscosity modifiers (e.g. sugars and modified celluloses) in the sample to slow down the reagent migration.

Preferably, the immobilized reagent in the detection zone is impregnated throughout the thickness of the carrier in the detection zone (e.g. throughout the thickness of the sheet or strip if the carrier is in this form). Such impregnation can enhance the extent to which the immobilized reagent can capture any analyte or labeled reagent, present in the migrating sample.

Reagents can be applied to the porous carrier material in a variety of ways. Various “printing” techniques have previously been proposed for application of liquid reagents to carriers, e.g. micro-syringes, pens using metered pumps, direct printing and ink-jet printing, and any of these techniques can be used in the present context. To facilitate manufacture, the carrier (e.g. sheet) can be treated with the reagents and then subdivided into smaller portions (e.g. small narrow strips each embodying the required reagent-containing zones) to provide a plurality of identical carrier units.

An assay based on the above principles can be used to determine a wide variety of analytes by choice of appropriate specific binding reagents. The analytes can be, for example, proteins, haptens, immunoglobulins, hormones, polynucleotides, steroids, drugs, infectious disease agents (e.g. of bacterial or viral origin) such as Streptococcus, Neisseria and Chlamydia. Sandwich assays, for example, may be performed for analytes such as carboxylated osteocalcin along with other markers that disclose various aspects of blood chemistry that affect blood clotting and/or heart disease.

Those skilled in the art will recognize that the test strip embodiment described above is not the sole test strip embodiment contemplated, and that the embodiment may take the form of several different versions. The immunoassay test strip embodiment may be an embodiment similar to that taught in U.S. Pat. Nos. 4,168,146; 4,678,757; 4,806,311; 4,837,168; and 4,891,313, by no means an exhaustive list, which are configured to be employed in accord with the teachings herein to determine the presence of a Vitamin K marker in a patient's body fluid sample. Further, it will be readily apparent to those skilled in the art in view of the teachings herein that several different schemes for separating out red blood cells from a patient's blood sample may be incorporated into the device if necessary depending on the particular constraints and sensitivity of the device.

Noted Considerations

The teachings of the references cited throughout the specification are incorporated herein in their entirety by this reference to the extent they are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

1. An apparatus for analyzing the concentration of a component in a body fluid sample, said apparatus comprising a housing configured to receive a disposable diagnostic reagent unit which exhibits a color change upon sensing said component in the body fluid sample, said apparatus further comprising:
a. a light source and light sensor for measuring light emanating from said source and reflected by reagent chemistry in said unit and having an optical characteristic proportional to the component of a liquid to be measured after transporting said liquid to said reagent; whereby an electrical signal responsive to a change in said reagent chemistry is generated, and therefore also to the component to be measured;
b. a microprocessor for processing said generated electrical signal; and
c. a display responsive to said processed signal for providing a visual readout representative of the analysis on said;
wherein said disposable diagnostic reagent unit comprises said reagent chemistry to detect a vitamin K marker in said body fluid sample.

2. A method of determining the proper dosage for an anticoagulant comprising the steps of measuring the vitamin K nutritional status of a patient; and modulating the dosage of an anticoagulant based on the patient's Vitamin K nutritional status.

3. The method of claim 2, wherein modulation of anticoagulation dosages comprises increasing dosages, decreasing dosages or maintaining dosages based on the expected effect of certain nutritional values for vitamin K in the patient, as well as a patient's diet patterns.

4. An apparatus analyzing the concentration of a component of a body fluid sample, said apparatus comprising a housing configured to receive a disposable diagnostic reagent unit which produces a signal upon detecting said component in the liquid, said apparatus further comprising:
a. electromechanical and software components for receiving and processing said signal from said disposable diagnostic reagent unit; and
b. a display responsive to said processed signal for providing a visual readout representative of the analysis on said;
wherein said disposable diagnostic reagent unit comprises said reagent chemistry to detect a vitamin K marker in said body fluid sample.

5. The apparatus of claim 4, wherein said electromechanical components comprise a microprocessor.

6. The apparatus of claim 4, wherein said signal produced by said disposable diagnostic reagent unit is an electrical signal produced by electrochemically detecting said component in said body fluid sample.

7. The apparatus of claim 6, wherein said electrochemically detecting comprises sensing changes in voltage produced by the reagent chemistry and interaction with said component in said body fluid sample.

8. A diagnostic reagent unit for analyzing a liquid sample suspected of containinig a Vitamin K marker, said device comprising the following components:
(1) a liquid sample application member;
(2) a liquid sample receiver; said receiver comprising a mobilizable labeled specific binding reagent for binding to analyte in said sample; and
(3) a dry porous carrier strip downstream of said liquid sample receiver, said carrier strip including a detection zone comprising an unlabeled immobilized specific binding reagent for binding to said analyte, said mobilizable labeled reagent being freely soluble or dispersible in liquid sample and transported by said liquid sample from said liquid receiver to detection zone;
wherein said analyte is a Vitamin K marker.

9. The diagnostic reagent unit of claim 8, wherein said Vitamin K marker is carboxylated osteocalcin.

10. The diagnostic reagent unit of claim 8, wherein said mobilizable labeled specific binding reagent is a labeled anti-carboxylated osteocalcin antibody.

11. The diagnostic reagent unit of claim 8, wherein said immobilized specific binding reagent is an anti-carboxylated osteocalcin antibody.

12. The diagnostic reagent unit of claim 8, wherein the presence of vitamin K marker produces a detectable signal corresponding to the concentration of the vitamin K marker.
